(19) United States
(12) Patent Application Publication
(10) Pub. No.: US 2013/0165942 A1
(43) Pub. Date: Jun. 27, 2013

(54) Length-Adjustable Vertebral Body Balloon

(71) Applicants: Francisca Tan-Malecki, West Lake Village, CA (US); Paul S. Maguire, Hope Valley, RI (US); Scott Olson, Princeton, MN (US); Robert Garryl Hudgins, Monticello, MN (US)

(72) Inventors: Francisca Tan-Malecki, West Lake Village, CA (US); Paul S. Maguire, Hope Valley, RI (US); Scott Olson, Princeton, MN (US); Robert Garryl Hudgins, Monticello, MN (US)

(21) Appl. No.: 13/719,676

(22) Filed: Dec. 19, 2012

Related U.S. Application Data
(60) Provisional application No. 61/579,574, filed on Dec. 22, 2011.

(51) Int. Cl. A61B 17/88

(52) U.S. Cl. CPC A61B 17/8855; A61B 17/8819
USPC 606/94; 606/86 R

(57) ABSTRACT

A length-adjustable vertebral body balloon catheter in which the location of the balloon can now be manipulated once an access cannula has been placed in the vertebral body. The device includes a collet such as a Touhy-Borst valve that allows the clinician

### CONTINUING DATA

This application claims priority from co-pending application U.S. Ser. No. 61/579,574, filed Dec. 22, 2011, entitled “Length-Adjustable Vertebral Body Balloon” (Docket No. DEP6433USPSP), the specification of which is incorporated by reference in its entirety.

### BACKGROUND OF THE INVENTION

In vertebral body augmentation, the surgeon or clinician seeks to treat a compression fracture of a vertebral body by injecting bone cement such as PMMA into the fracture site. In certain procedures called “vertebroplasty”, the cement is injected into the fracture site without first pretreating the site. In another procedure called “kyphoplasty”, a balloon is first delivered to the fracture site and then expanded to create a large void in the bone. Cement is then injected into the void. It has been contemplated that the creation of the large void in kyphoplasty enhances the safety of the cement injection step. It has also been contemplated that performing kyphoplasty may further lead to some height restoration of the fractured vertebral body.

The following patent documents are considered relevant to the kyphoplasty art: EP 1459689; WO09058831; WO06088649; EP1073371; EP2108323; WO09149108; WO08011410; EP0836435; EP1272131; WO08026888; WO080137428; EP110426081; EP1509175; EP1464293; EP1463464; EP1257217; WO09146428; EP0741547; WO07008568; WO09065085; and WO07067726.

The following documents disclose cardiovascular balloon catheters equipped with Touhy-Borst valves: U.S. Pat. No. 4,886,507; U.S. Pat. No. 5,203,774; and U.S. Pat. No. 5,389,090.

### SUMMARY OF THE INVENTION

It has been observed that in conventional kyphoplasty systems having relatively elastic balloons, there is currently an inability to control the longitudinal expansion of the balloon (i.e., expansion along the longitudinal axis of the catheter). Accordingly, the shape of the balloon in the vertebral body can not be finely manipulated. This inability may pose a problem in patients having relatively weak or porous bone, as the elastic balloon may undesirably expand in the longitudinal direction to the point where it reaches the anterior wall of the vertebral body and poses the danger of breach. Therefore, it is an object of the present invention to finely manipulate the shape of the balloon in the longitudinal direction so as to minimize undesirable elongation. It is a particular object of the present invention to finely manipulate the shape of the balloon in the longitudinal direction after balloon expansion has begun so as to minimize any further elongation.

It has further been noticed that in kyphoplasty systems, it is possible for the proximal end of the access cannula to have a fixed luer lock connection that may be mated with a luer lock connection fixedly attached to the tube of the balloon catheter. Because both fittings of the luer lock are fixedly attached, the final location of the balloon is dictated by the depth to which the access cannula is inserted into the vertebral body. Accordingly, the location of the balloon in the vertebral body can not be manipulated once the access cannula has been placed in the vertebral body. It is believed that this inability to adjust the depth of the balloon after cannula placement presents challenges to the surgeon/clinician. For example, it is believed desirable for the surgeon/clinician to be able to adjust the location of balloon in the vertebral body after the balloon has been placed in the vertebral body and viewed on the x-ray.

Therefore, it is an object of the present invention to provide for adjustment of the location of the balloon after cannula placement, so that the clinician can adjust the depth of balloon insertion even after the balloon has been placed, luer locked to cannula and viewed on the x-ray.

The present invention relates to a length-adjustable vertebral body balloon catheter. In some embodiments, the location of the balloon can be manipulated after the access cannula has been placed. In some embodiments, the location of the balloon can be manipulated even after balloon expansion has begun.

In preferred embodiments, the proximal end of the catheter is fitted with a fluid-tight collet such as a Touhy-Borst valve and the inner rod of the catheter is made to float freely within the outer tube. When the Touhy-Borst valve is in its open configuration, the rod can be moved and the location and/or distal extremity of the balloon can thereby be adjusted. Once the desired location and/or distal extremity of the balloon has been achieved, the Touhy-Borst valve can be closed, thus locking the position of the rod (and thereby the balloon and balloon length) in its desired location.

Therefore, in accordance with the present invention, there is provided a catheter system comprising:
a) an introducer comprising i) a stylet adapted to pierce a vertebral body and ii) an access cannula for accessing the vertebral body, the cannula having a proximal end portion and a distal end portion, wherein the stylet is received in the cannula;
b) an expansion catheter comprising i) an inflatable device having a distal end portion and an proximal end portion, ii) a rod having a distal end portion and a proximal end portion, and iii) an outer tube having a proximal end portion and a distal end portion, wherein the proximal end portion of the inflatable device is attached to the distal end portion of the outer tube, and wherein the distal end portion of the rod is attached to the distal end portion of the inflatable device;
c) a hub having a bore having a distal end portion attached to the proximal end portion of the outer tube and a proximal end portion,
d) a collet connected to the proximal end portion of the hub, the collet forming a bore;
wherein the rod is received in the outer tube,
wherein the outer tube is receivable in the access cannula, and
wherein the proximal end portion of the rod is received in the bore of the collet.

### DESCRIPTION OF THE FIGURES

FIG. 1 discloses a cross-section of the introducer of the present invention.

FIG. 2 is a perspective view of a device of the present invention within a bone cannula.

FIGS. 3a-3c are side views of another embodiment of the device of the present invention having an inner guidewire.

FIG. 4 is a cross-sectional view of the distal end portion of an embodiment of the present invention having a dual tube configuration.

FIGS. 5a-5c are perspective and cross-sectional views of the proximal end portion of an embodiment of the present invention.

FIG. 6 is a cross-sectional views of an embodiment of the Touhy-Borst Adapter of the present invention.

### DETAILED DESCRIPTION OF THE INVENTION

For the purposes of the present invention, a “rod” will be considered to include both a guidewire alone and an inner catheter tube adapted to receive a guidewire. The terms “inflatable device” and “expandable device” are used interchangeably.

In general, the invention relates to the therapeutic treatment of a fractured bone having with a cortical wall surrounding an interior bone volume, such as a vertebral body. The invention is carried out through the use of a balloon-like inflatable device that is delivered percutaneously into the bone. In a first step, the inflatable device is delivered in an initial deflated configuration. In a second step, the inflatable device expands to an inflated configuration (preferably having a predetermined shape) to create a cavity or void within the interior bone volume. Preferably, this inflation also at least partially restores the original position of the outer cortical wall of the bone. The inflated device is then deflated and removed from the bone. Lastly, bone cement is injected into the void to achieve fracture fixation.

In preferred embodiments of the general method described above, the Touhy-Borst valve is manipulated to allow the clinician to adjust and lock the depth of insertion of the rod, thereby constraining the longitudinal expansion of the balloon. This longitudinal constraint allows the clinician to desirably fix the shape and location of the inflated balloon.

Percutaneous access into the vertebral body can be via an ipsilateral, posteriolateral or transpedicular access route. The balloon is delivered in an initial, uninflated configuration through the percutaneous access. Expansion of the balloon compresses a portion of the cancellous bone to create a cavity or void within the cancellous bone and may potentially restore at least some of the height of the vertebral body that was lost during fracture.

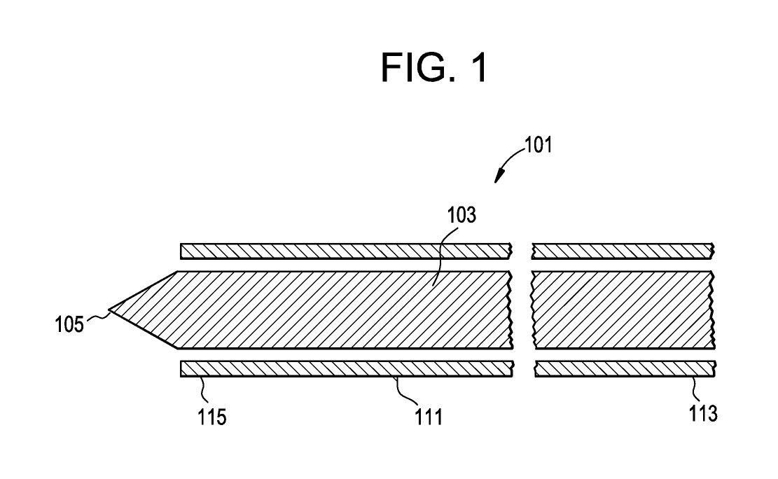

Now referring to FIG. 1, there is provided an introducer 101 comprising i) a stylet 103 having a distal tip 105 adapted to pierce a cortical wall of a vertebral body and ii) an access cannula 111 for accessing the vertebral body, the cannula having a proximal end portion 113 and a distal end portion 115, wherein the stylet is received in the cannula.

In some embodiments, the stylet may be threaded so as to form a drill. In some embodiments, the stylet may have a handle attached to its proximal end portion so as to improve its manual ease of use or control during insertion or rotation.

In some embodiments, the access cannula is made of very stiff material, such as a metallic or ceramic material. In some embodiments, the inner diameter of the access cannula is at least as large as 12 gauge, preferably at least as large as 11 gauge, more preferably at least as large as 10 gauge.

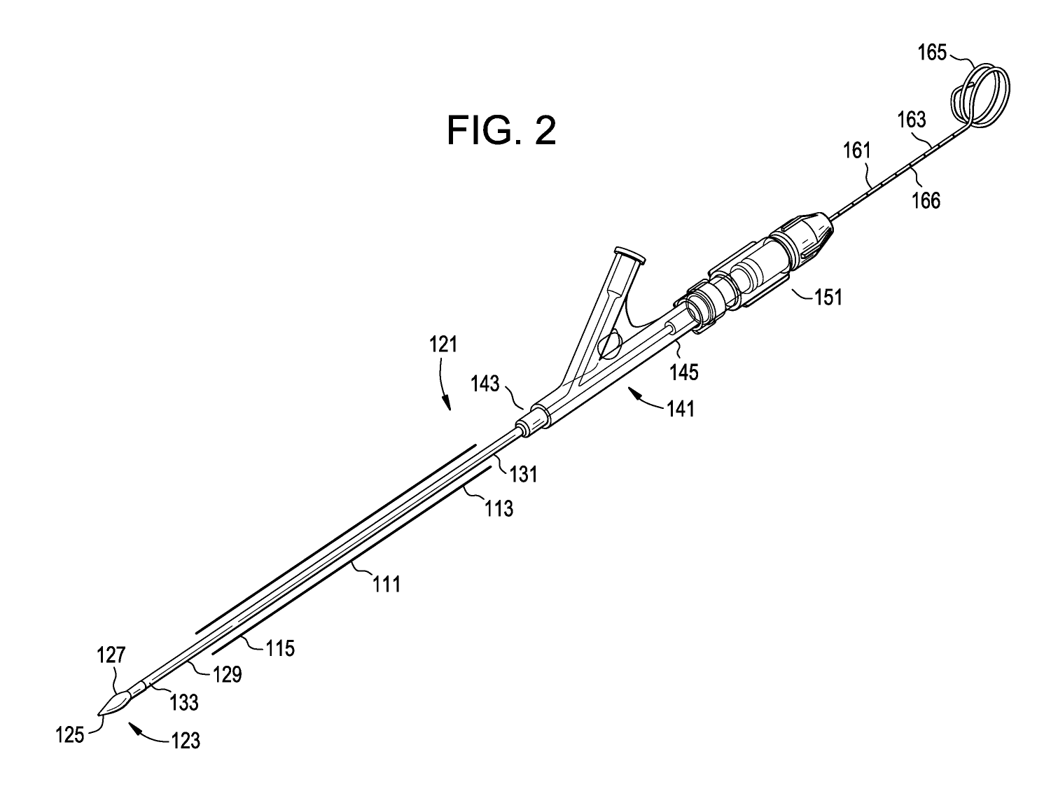

Now referring to FIG. 2, there is provided a catheter system comprising:
a) an introducer comprising an access cannula 111 for accessing the vertebral body, the cannula having a proximal end portion 113 and a distal end portion 115, wherein the stylet is received in the cannula;
b) an expansion catheter 121 comprising i) an inflatable device 123 having a distal end portion 125 and an proximal end portion 127, ii) a rod 161 having a proximal end portion 163 forming a handle 165 and iii) an outer tube 129 having a proximal end portion 131 and a distal end portion 133, wherein the proximal end portion of the inflatable device is attached to the distal end portion of the outer tube;
c) a hub 141 having a bore and having a distal end portion 143 attached to the proximal end portion of the outer tube and a proximal end portion 145,
d) a collet 151 connected to the proximal end portion of the hub, the collet forming a bore,
wherein the rod is received in the outer tube,
wherein the outer tube is receivable in the access cannula, and
wherein the proximal end portion of the rod is receivable in the bore of the collet.

In use, the patient's targeted vertebral body is first percutaneously accessed by the introducer. The stylet component of the introducer is withdrawn and the device (which comprises the expansion catheter, hub and collet) is delivered into the vertebral body via the remaining cannula, with the collet in an open configuration.

In one embodiment wherein the device is fixedly attached to the cannula via a luer connections on the proximal end portion of the cannula and the distal end of the hub, the handle of the rod is then manipulated after luer fixation to adjust the distal end portion of the rod. The, the balloon is inflated to the desired shape to form a cavity, deflated, and the device is withdrawn. Lastly, cement is injected into the cavity.

In another embodiment, the balloon is inflated with the collet valve open until the distal end of the balloon reaches a desired location. The collet is then closed to lock the distal end portion of the balloon, and inflation is resumed until the balloon achieves its desired shape. The balloon is then deflated, and the device is withdrawn, leaving behind a cavity. Lastly, cement is injected into the cavity.

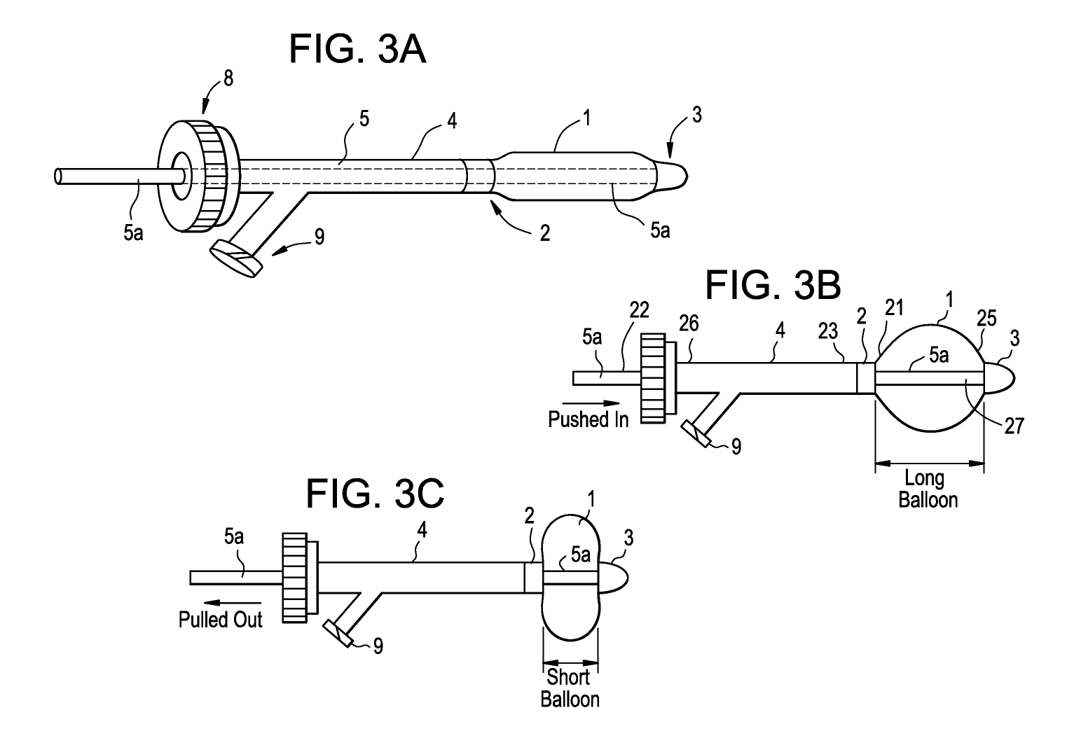

Now referring to FIGS. 3a-6, which show various views of the distal and proximal ends of the expansion catheter, the device of the present invention may preferably have the following components: Inflatable body 1; Radiopaque markers 2 and 3; Outer catheter tube 4; Inner catheter tube 5; Guide wire 6; Y-Hub 7; Collet 8; Saline injection port of catheter 9; and Proximal end port of catheter 10.

In some embodiments, the outer tube of the expansion catheter and the Y-Hub can be intergrated into a single unitary component. As an example thereof, and now referring to FIGS. 3a-3c, there is provided a device comprising:
a) an expansion catheter comprising i) an inflatable device 1 having a distal end portion 25 and an proximal end portion 21, ii) a guidewire 5a having a distal end portion 27 and a proximal end portion 22, and iii) an outer tube 4 having a proximal end portion 26 and a distal end portion 23, wherein the proximal end portion of the inflatable device is attached to the distal end portion of the outer tube, and wherein the distal end portion of the guidewire is attached to the distal end portion of the inflatable device; and
b) a collet 8 connected to the proximal end portion of the hub, the collet forming a bore;
wherein the guidewire is received in the outer tube,
wherein the outer tube is receivable in an access cannula, and
wherein the proximal end portion of the rod is received in the bore of the collet.

In some embodiments, the catheter has a guidewire-in-tube design so that the inflatable body is attached to the distal ends of the respective rod and the tube. In some embodiments, and now referring to FIGS. 3a-3c, the proximal end 21 of the inflatable body 1 is connected to the distal end portion 23 of a outer tube and the distal end portion 25 of the inflatable body is connected to the distal end portion 27 of the guidewire, which extends beyond the distal end portion of the tube. The guidewire floats 5a freely within the balloon and outer tube, and is locked only by the Touhy-Borst adapter.

FIG. 3b shows the device with an expanded balloon, wherein the length of the balloon is relatively long. In this embodiment, the guidewire has been pushed into the outer tube. FIG. 3c shows the device with an expanded balloon, wherein the length of the balloon is relatively short. In this embodiment, a portion of the guidewire has been pushed out of the outer tube.

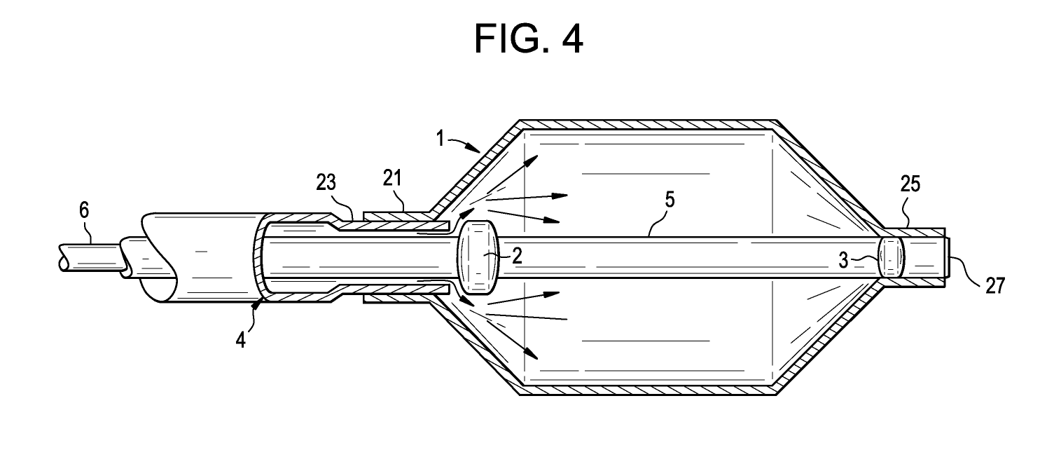

In some embodiments, and now referring to FIG. 4, the expansion catheter has a dual tube design so that the inflatable body is attached to the distal ends of a pair of coaxial catheter tubes, whereby the inner tube 5 extends beyond the distal end portion 32 of the outer catheter tube 4. In some embodiments, the proximal end portion 21 of the inflatable body is connected to the distal end portion 23 of the outer tube and the distal end portion 25 of the expandable body is connected to the distal end portion 27 of the inner catheter tube. The inner tube floats freely within the balloon and outer tube, and is locked only by the Touhy-Borst adapter (not shown). The device further includes a distal radiopaque marker 3 located in the distal end portion of the inner tube, while a more proximal portion of the inner tube carries a second radiopaque marker 2. The distal end portion of the outer tube may be narrowed (as in FIG. 4) in order to more easily accommodate the balloon within the profile of the expansion catheter.

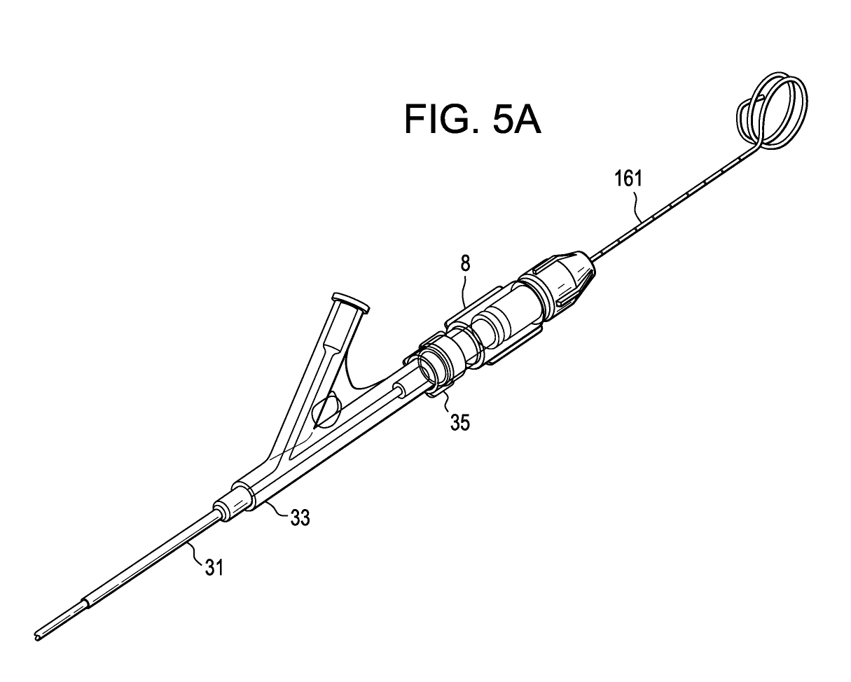

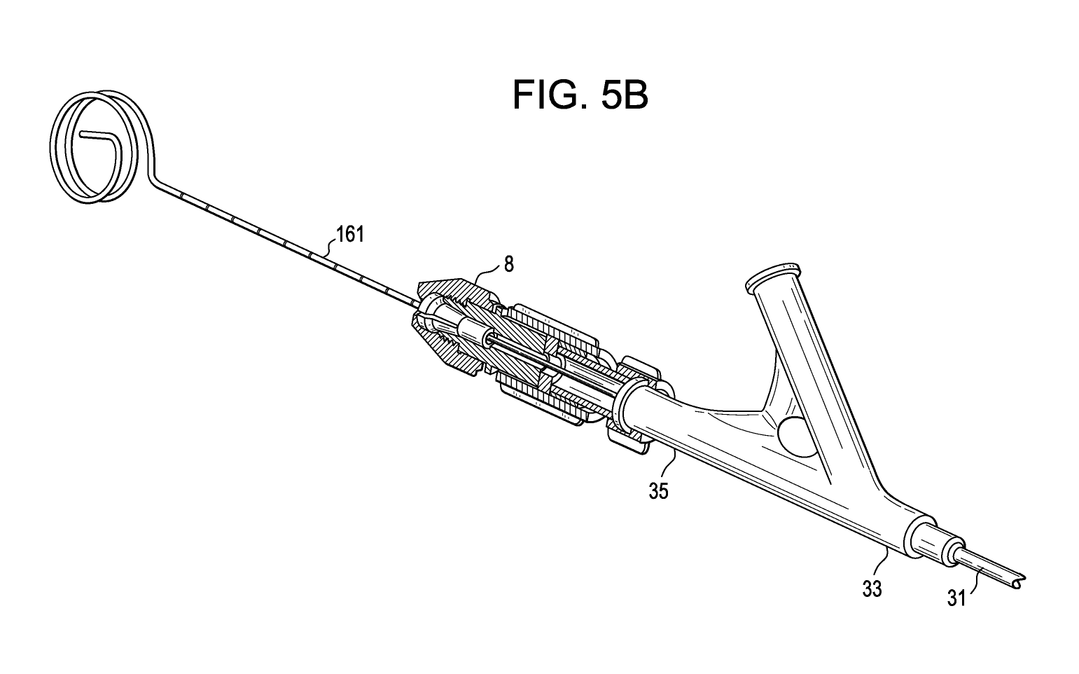

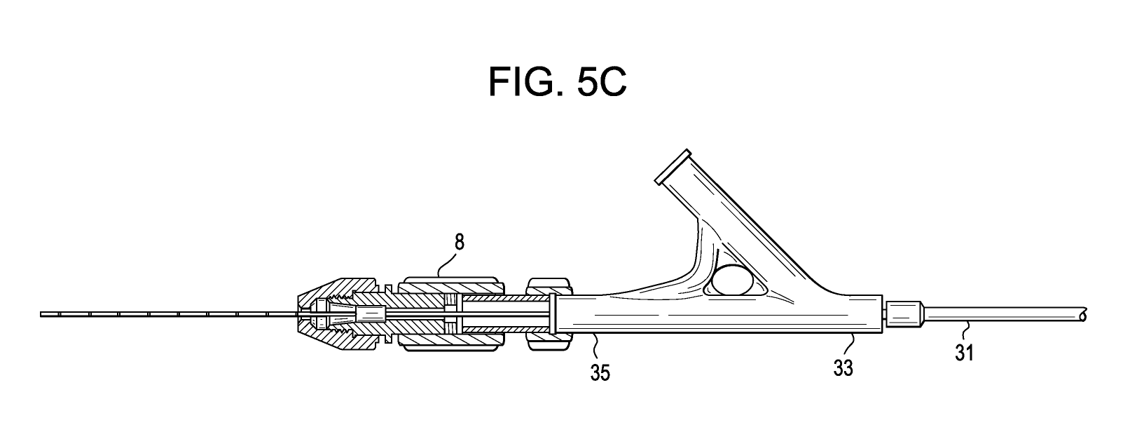

FIG. 5a discloses a perspective view of a portion of the device, showing a proximal end portion 31 of the outer tube attached to a distal end portion 33 of the Y-hub. FIG. 5 further shows the proximal end portion 35 of the Y-Hub attached to the collet 8, and guidewire 161 received in the collet.

FIG. 5b discloses another perspective view of a portion of the device, showing details of the collet 8 in cross-section.

FIG. 5c discloses a side view of a portion of the device, showing details of the collet 8 in cross-section.

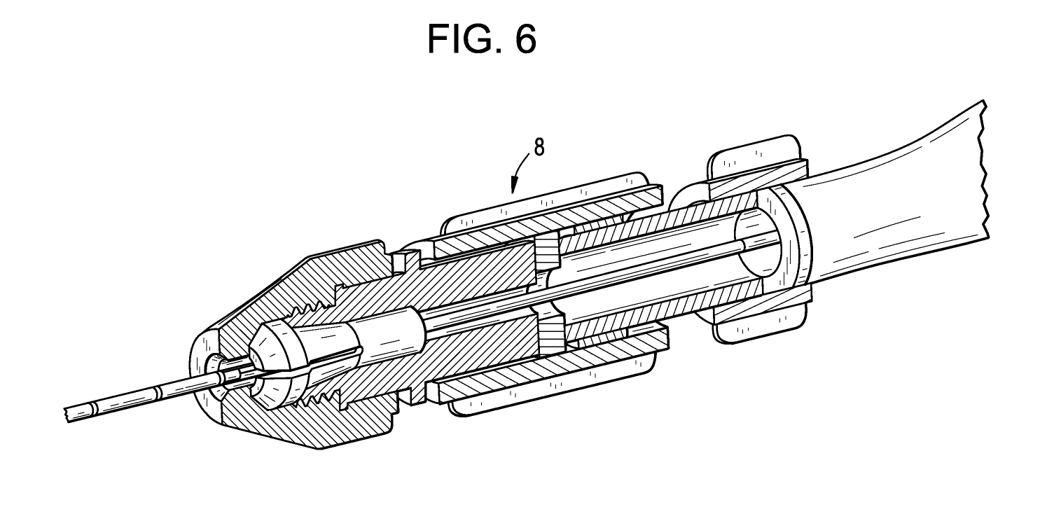

FIG. 6 discloses a perspective view of the collet 8 portion of the device, wherein the collet is a Touhy-Borst valve. A portion of the collet is provided in cross-section in order to details the mechanical details of the valve.

In some embodiments, the inflatable body is made of an elastomeric material, such as a polyurethane. It is typically made to expand to a desired predetermined volume by subjecting the inflatable body to pressure and heat. The inflatable body of the present invention is preferably a balloon. In some embodiments, the inflatable body is elastic. In some embodiments, the inflatable body is inelastic and so expands to a predetermined volume (unless truncated by the use of the present invention). In some embodiments, the volume of the expanded balloon is at least 3 cc. In others, it is at least 5 cc. In embodiments directed to vertebral bodies, the volume is preferably between 3 cc and 8 cc. In some embodiments, the length of the unexpanded balloon (along the outer tube longitudinal axis) is between 5 mm and 20 mm. In some embodiments, the length of the expanded balloon (along the outer tube longitudinal axis) is between 10 and 30 mm. In some embodiments, the major diameter of the expanded balloon is between 10 and 30 mm in an unconstrained state. In use, the major diameter of the expanded balloon in constraining bone is between 10 and 20 mm.

In general, saline may be injected into the inflatable body through a fitting or port 9 located in the hub in order to expand the inflatable body and create the desired cavity. In some embodiments, the saline may be injected in predetermined amounts. The saline may include a radiographic contrast agent to improve the visibility of the balloon on x-ray after or during inflation.

In some embodiments, the outer catheter tube is made of i) an outer layer of an elastomeric material, preferably of the same elastomeric material as the inflatable body in order to promote binding therebetween, and ii) an inner layer of a polyamide material (such as nylon or Kevlar) in order to promote strength. In some embodiments (as in FIG. 4), the distal end portion of the outer catheter tube tapers slightly to form a neck. This neck minimizes the extra thickness added to the expansion catheter profile by the balloon at this bonding location.

In some embodiments, the inner catheter tube is made of an elastomeric material. In some embodiments, the inner tube comprises the same elastomeric material as the inflatable body in order to promote binding therebetween.

In most embodiments wherein the rod comprises a guidewire within an inner tube, the function of the guidewire is to provide a stiffening element that mechanically supports the inner tube. In some embodiments, the function of the guidewire is to occlude the bore of the inner tube, and thereby prevent fluid leakage therethrough. The guidewire preferably extends through the inner tube up to the junction of the inner tube and the balloon. In some embodiments, the guide wire is made of a medical grade stainless steel and has a proximal handle for ease of manual manipulation. In some embodiments, the proximal end portion of the guidewire fits into the bore of the Touhy-Borst adapter to adjustably position the balloon.

In some embodiments, the rod comprises a guidewire alone. In these embodiments, as in FIGS. 3a-3b, the inner catheter tube is eliminated and the distal end portion of the guidewire bonds directly to the distal end portion of the inflatable device.

Now referring to FIG. 2, in some embodiments, radiographic markings 166 are provided at equal increments down the length of the guide wire. In one embodiment, these markings are spaced about 5 mm apart. Providing markings on the guide wire increases the user's awareness of the cavity being created in the vertebral body. In addition to aiding fluoroscopic imaging, the guide wire markings can serve as an external guide to the longitudinal growth of the balloon, which also correlates to the length of the cavity. Moreover, the markings help the user set the length of balloon if the user wants the balloon to expand to a specific length.

Now referring to FIG. 4, in other embodiments, radiopaque markers may be placed upon the portion of the rod located within the balloon volume. The purpose of these radiopaque markers is to provide the clinician with an indication of the location and size of the balloon in the bone under x-ray. In general, the markers extending from the outer tube upon the distal end portion of the rod substantially indicate the distal and proximal locations of the expanded portion of the balloon. In some embodiments in which the rod comprises a guidewire received in an inner tube, a distal-most radiopaque marker is placed within the bore of the inner tube, just distal of the distal end of the guide wire. Its location is preferably near the junction of the inner tube and the balloon. The more proximal radiopaque markers located within the balloon may be placed around (swaged over) the inner tube at graduated increments. The markers are generally made of medical grade stainless steel.

Now referring to FIG. 3a, the outer tube 5 proximally attaches directly to a fitting of the Touhy-Borst Adapter 8. The proximal portion of the outer tube further includes a second more distal opening forming a second port 9 that is adapted for fitting to a saline injector.

In other embodiments, and now referring to FIGS. 5a-5c, the proximal portion 31 of the outer tube attaches to a distal portion 33 of a hub. The hub is preferably a thermoplastic material, such as polycarbonate, with a strain relief made of a thermoplastic elastomer, such as PEBAX. The hub may be transparent and injection molded onto the outer catheter tube. The hub is preferably a Y-hub that bifurcates proximally into the two separate proximal ports. These ports respectively provide distal passage therethrough of both a) the guidewire extending from the collet (such as a Touhy-Borst Adapter) and b) saline delivered from a saline injection device

Preferably, the guidewire extends through the proximal port that is disposed substantially linearly with respect to the distal end portion of the Y-hub.

Preferably, the collet is fluid tight to avoid fluid leakage from the saline injector or saline-filled balloon. In preferred embodiments, the collet is a Touhy-Borst Adapter (TBA). Preferably, as shown in FIGS. 5a-5c, the TBA 8 is mounted on the proximal end portion 35 of the Y-Hub.

Now referring to FIGS. 3b-3c, there are provided side views of the expansion catheter of the present invention. FIG. 3b discloses the inflatable device at a first longer length. FIG. 3c discloses the inflatable device at a second shorter length. Owing to the easy adjustability affording by the collet, the user can adjust the extent to which the rod extends from the outer tube. Accordingly, the user can adjust the length of the inflatable device between these two lengths after the expansion catheter has been inserted into the vertebral body, and even after balloon expansion has begun.

In general, the catheter system of the present invention possesses many benefits. First, it provides an opening within the body through minimally invasive means. It has a balloon bonded to a catheter shaft. Saline passes through the second port of the Y-hub and through the catheter shaft to inflate the balloon. After the desired expansion has been achieved, withdrawal of the saline deflates the balloon and allows the balloon to be withdrawn from the patient through the cannula.

Generally, the present invention comprises a catheter system adapted assist in the fixing of a fractured vertebral body, wherein an outer tube of the catheter is fitted with a manipulable locking system for adjusting the length of a rod extending therefrom. This locking system, which can include a collet such as a Touhy-Borst adapter, allows the user to exercise more selective control over balloon expansion. When the collet is in its open (or “unlocked”) position, the rod can move freely longitudinally and so the balloon can inflate in an unconstrained manner. When the collet is in its closed (or “locked”) position, it locks down on the rod and substantially fixes the length of the balloon. This allows the clinician to select the length of the cavity to be created in vertebral body, and to do so after inserting the balloon into the bone and even after inflation as begun. It also allows the user to fix the longitudinal growth of balloon while continuing to expand the balloon radially.

In some embodiments, the balloon is proximally bonded to the outer tube with the rod embedded into the distal end portion of the balloon. The Y-hub extending from the proximal end portion of the outer tube preferably is attached to a Touhy-Borst Adapter (TBA) or a locking collet that allows the rod to pass through and out of the bore of the outer tube. When the TBA /collet is in the open/unlocked position, the rod can move freely through the TBA. In some embodiments, a seal distal to the TBA/collet of the Y-hub maintains inflation pressure for balloon expansion but allows for guidewire movement.

When the TBA/collet is open, the rod is unlocked and the balloon dimensions can be manipulated. In one method, the user can predetermine the length of the cavity to be created in the vertebral body by locking down the length prior to balloon insertion.

In another method, the user initially inflates the balloon with the collet open, then closes the collet at some point during inflation. If the balloon grows longitudinally more than desired, the user can select to close the collet, thereby locking the expanded length of the balloon while continuing balloon inflation in only the radial direction.

A third method of the present invention uses the collet in the creation of a secondary cavity within the vertebral body. The creation of the first cavity involves the user leaving the collet open to allow the balloon to freely expand to any dimension passively allowed within the vertebral body. It is believed that the extent of actual expansion in this “open” collet condition would depend upon the bone density, fracture location and other anatomical factors. At the end of the inflation, the user then closes the TBA to lock the rod. Next, the user deflates the balloon and then reinflates the same balloon to create a second cavity in the same vertebral body at a set length with the Touhy Borst Adapter/collet still closed. This method thus creates a second cavity having exactly the same length as the first cavity, thereby providing a measure of symmetry in a bilateral procedure. The user can either remove the balloon from the vertebral body or keep the balloon within the vertebral body to lock the collet and set balloon length for the second inflation.

In some embodiments, the distal end of the guidewire is connected to the distal end of the balloon. This feature is one way by which the device of the present invention is distinguished from a conventional cardiovascular balloon.

In some embodiments, the distal end of the inner tube is sealed. This feature is another way by which the device of the present invention is distinguished from a conventional cardiovascular balloon.

In a general method of using the present invention, a first tool (such as an introducer needle) having a bore (such as an access cannula) is introduced into the bone through a percutaneous access path. Next, a second tool (such as the balloon catheter) is delivered into the bone through the percutaneous access path by passing through the bore of the first tool. Next, the inflatable device of the second tool undergoes expansion in the interior bone volume, thereby compacting the interior bone volume and creating a cavity (or “void”) within the interior bone volume. The second tool is then deflated and removed from the bore of the first tool. Lastly, bone cement is injected into the cavity.

In some embodiments, there is provided a method of augmenting a fractured vertebral body of a patient, comprising the steps of:
a) inserting an introducer comprising a stylet and an access cannula having a proximal end portion and distal end portion into the fractured vertebral body;
b) withdrawing the stylet from the patient;
c) inserting an expansion catheter comprising a balloon through the proximal end portion of the access cannula so that the balloon extends from the distal end portion of the access cannula;
d) opening a Touhy-Borst valve attached to the proximal end of the catheter;
e) adjusting the position of the balloon within the vertebral body;
f) tightening the Touhy-Borst valve to lock a position of the balloon catheter;
g) expanding the balloon to create a void space within the fractured vertebral body;
h) deflating the balloon;
i) removing the balloon; and
j) injecting a bone cement into the void space of the fractured vertebral body.

In preferred embodiments, the bone is a vertebral body. In some embodiments, the vertebral body is fractured. In some embodiments, the vertebral body has a tumor. The present invention can be used in treating a bone such as a vertebral body that is intact but predisposed to fracture or collapse, such as an osteoporotic bone or a vertebral body that is adjacent a cemented vertebral body.

In another aspect of the present invention, bilateral balloons are deployed in the same vertebral body. The first balloon is inserted into the interior or a vertebral body through a first access path (such as a first pedicle). The second balloon is inserted into the same vertebral body through a different access path in the cortical bone that is different than the first access path (such as a second pedicle). The first and second access paths can be two different ipsilateral access paths, two different transpedicular access paths, or one ispsilateral posterolateral path and one transpedicular access path. The two balloons so inserted can then be inflated either serially or simultaneously.

Preferred bone pastes include bone cements (such as acrylic-based bone cements, such as PMMA-based bone cements), pastes comprising bone particles (either mineralized or demineralized or both; and either autologous, allogenic or both), and ceramic-based bone cements (such as HA and TCP-based pastes).

In some embodiments, the injection of bone cement into the cavity created by the expanded balloon is carried out with the CONFIDENCE™ injector and bone cement marketed by DePuy Spine, Raynham, Mass.

We claim:

1. A catheter system comprising:
a) an introducer comprising i) a stylet adapted to pierce a vertebral body and ii) an access cannula for accessing the vertebral body, the cannula having a proximal end portion and a distal end portion, wherein the stylet is received in the cannula;
b) n expansion catheter comprising i) an inflatable device having a distal end portion and an proximal end portion, ii) a rod having a distal end portion and a proximal end portion, and iii) an outer tube having a proximal end portion and a distal end portion, wherein the proximal end portion of the inflatable device is attached to the distal end portion of the outer tube, and wherein the distal end portion of the rod is attached to the distal end portion of the inflatable device;
c) a hub having a bore having a distal end portion attached to the proximal end portion of the outer tube and a proximal end portion,
d) a collet connected to the proximal end portion of the hub, the collet forming a bore;
wherein the rod is received in the outer tube,
wherein the outer tube is receivable in the access cannula, and
wherein the proximal end portion of the rod is received in the bore of the collet.

2. The system of claim 1 wherein the inflatable device is expandable to at least 3 cc.

3. The system of claim 1 wherein the inflatable device is expandable to at least 5 cc.

4. The system of claim 1 wherein the inflatable device is a balloon.

5. The system of claim 1 wherein the inflatable device is elastic.

6. The system of claim 1 wherein the stylet is threaded.

7. The system of claim 1 wherein the collet is a Touhy-Borst valve.

8. The system of claim 1 wherein the proximal end portion of the rod has a handle.

9. The system of claim 1 wherein the stylet comprises a distal tip adapted to pierce a cortical wall of the vertebral body.

10. The system of claim 1 wherein the rod comprises a guide wire.

11. The system of claim 1 wherein the rod comprises an inner tube adapted for reception of a guidewire.

12. The system of claim 1 wherein the access cannula has an inner diameter that is at least as large as 12 gauge.

13. The system of claim 1 wherein the access cannula comprises a metallic or ceramic material.

14. The system of claim 1 wherein the hub further includes a port adapted for reception of a fluid injector.

15. A method of using a balloon catheter comprising a balloon having a proximal end portion attached to an outer tube and a distal end portion attached to a rod received within the outer tube, the proximal and distal end portions defining a balloon length therebetween, comprising the steps of:
a) inserting an access cannula having a proximal end portion into a fractured vertebral body;
b) inserting the balloon through the access cannula with the balloon fixed at a first balloon length; and
c) adjusting the rod to produce a second balloon length.

16. The method of claim 15, wherein a collet is attached to the proximal end portion of the outer tube, and wherein the first length of the balloon is fixed by the collet in a closed position locking the rod.

17. The method of claim 15 wherein a collet is attached to the proximal end portion of the outer tube, and wherein the adjustment step is carried out with the collet in an open position.

18. The method of claim 15, wherein a collet is attached to the proximal end portion of the outer tube, wherein the first length of the balloon is fixed by the collet in a closed position locking the rod, and wherein the adjustment step is carried out with the collet in an open position.

19. The method of claim 15, wherein a collet is attached to the proximal end portion of the outer tube, the method comprising the further step of:
d) locking the adjusted rod to the collet to fix the balloon at the second length.

20. The method of claim 19 further comprising the step of:
e) expanding the balloon, the balloon being at its second length to create a cavity in the vertebral body.

21. The method of claim 20 further comprising the step of:
f) deflating the balloon

22. The method of claim 21 further comprising the step of:
g) removing the balloon from the vertebral body.

23. The method of claim 22 further comprising the step of:
h) injecting bone cement into the cavity.

24. A device for creating a cavity in a vertebral body, comprising:
a) an expansion catheter comprising i) an inflatable device having a distal end portion and an proximal end portion, ii) a rod having a distal end portion and a proximal end portion, and iii) an outer tube having a proximal end portion and a distal end portion, wherein the proximal end portion of the inflatable device is attached to the distal end portion of the outer tube, and wherein the distal end portion of the rod is attached to the distal end portion of the inflatable device;
b) a collet connected to the proximal end portion of the outer tube, the collet forming a bore;
wherein the rod is received in the outer tube,
wherein the outer tube is receivable in an access cannula, and
wherein the proximal end portion of the rod is received in the bore of the collet.
